(19) United States
(12) Patent Application Publication
(10) Pub. No.: US 2024/0352537 A1
(43) Pub. Date: Oct. 24, 2024

(54) COMPOSITION FOR DIAGNOSING PROSTATE CANCER BY USING CPG METHYLATION CHANGES IN SPECIFIC GENES, AND USE THEREOF

(71) Applicant: GENCURIX INC., Seoul (KR)

(72) Inventors: Sang Rae CHO, Seoul (KR); Young Ho MOON, Seoul (KR); Jinil HAN, Seoul (KR); Yun Young LEE, Seoul (KR)

(21) Appl. No.: 18/761,983

(22) Filed: Jul. 2, 2024

Related U.S. Application Data
(63) Continuation-in-part of application No. PCT/KR2023/000157, filed on Jan. 4, 2023.

(30) Foreign Application Priority Data
Jan. 5, 2022 (KR) 10-2022-0001649

(51) Int. Cl. C12Q 1/6886; C12Q 1/6851

(52) U.S. Cl. CPC C12Q 1/6886; C12Q 1/6851; C12Q 2600/154

(57) ABSTRACT

The present invention relates to a composition, a kit, a nucleic acid chip, and a method which make it possible to diagnose prostate cancer by detecting the methylation level of CpG sites of one or more genes selected from the group consisting of KLK4, HES5, TPM4, MOB3B, NNT, IL17RD, DTX4, NOL3, ADD3, EXT1, CPED1, and C2orf88, thus enabling an accurate and rapid diagnosis of prostate cancer, as well as the early diagnosis thereof.

### CROSS REFERENCE TO RELATED APPLICATIONS

This application is a continuation-in-part application of PCT/KR2023/000157 filed Jan. 4, 2023 and claiming the benefit of priority from Korean patent application KR 10-2022-0001649 filed Jan. 5, 2022, the entire disclosure of both applications is herein incorporated by reference.

### TECHNICAL FIELD

This application claims priority to Korean Patent Application No. 10-2022-000164, filed on Jan. 5, 2022, and the entire disclosure of which is incorporated herein by reference.

The present invention relates to a composition, kit, nucleic acid chip, and method for diagnosing prostate cancer by detecting the methylation level of CpG sites in one or more genes selected from the group consisting of KLK4, HES5, TPM4, MOB3B, NNT, IL17RD, DTX4, NOL3, ADD3, EXT1, CPED1, and C2orf88.

### BACKGROUND

Prostate cancer is generally known to have a good prognosis. However, the prognosis can vary greatly depending on the stage of cancer progression at the time of diagnosis, making early detection of prostate cancer crucial for improving patient survival rates. The diagnostic method using prostate-specific antigen (PSA), a representative tumor marker for prostate cancer, currently shows the best sensitivity among single prostate cancer tests and aids in the diagnosis and treatment of prostate cancer. Despite these results, PSA test has a low specificity, leading to the possibility of over-diagnosis and subsequent over-treatment. Therefore, the development of new diagnostic methods to replace the test is required.

Meanwhile, DNA methylation, a type of the epigenetic modifications, is associated with the expression (or suppression and induction) of disease-regulating genes within cells, and recently, cancer diagnostic methods measuring DNA methylation have been suggested. Specifically, since cancer-specific methylation can occur in pre-cancerous tissues, the detection of cancer-specific methylation is highly likely to be used in cancer diagnosis.

In particular, most of the markers developed so far are described as being capable of diagnosing cancers other than prostate cancer, indicating a need for the development of markers that can specifically diagnose prostate cancer for improved diagnostic efficiency.

For instance, PCT/IT2006/000237 describes methylation markers for ALDH1 A3, ARHGAP20, FLU, FOSL2, HES2, MAPI B, MDM2, NID1, NID2, PNUTL2, PTGER3, and PTPRM that can diagnose prostate cancer, but they are also described as being capable of diagnosing solid tumors such as gastric cancer and liver cancer.

Therefore, there is a need to develop effective prostate cancer-specific methylation markers that can predict the risk of prostate cancer.

### SUMMARY OF INVENTION

### Technical Problem

The inventors have discovered that specific CpG sites of certain genes are hypermethylated in prostate cancer and have developed compositions, kits, nucleic acid chips, and methods for diagnosing prostate cancer by detecting these methylation levels, thus completing the present invention.

Therefore, an object of the present invention is to provide a composition for diagnosing prostate cancer comprising an agent for measuring the methylation level of CpG sites of specific genes.

Another object of the present invention is to provide a composition for diagnosing prostate cancer consisting of an agent for measuring the methylation level of CpG sites of specific genes.

A further object of the present invention is to provide a composition for diagnosing prostate cancer essentially consisting of an agent for measuring the methylation level of CpG sites of specific genes.

Additionally, another object of the present invention is to provide a diagnostic kit for prostate cancer comprising a PCR primer pair for amplifying a fragment including the CpG sites of specific genes and a sequencing primer for pyrosequencing the PCR product amplified by the primer pair.

Another object of the present invention is to provide a nucleic acid chip for diagnosing prostate cancer, comprising a probe fixed to the kit that can hybridize under stringent conditions to a fragment including the CpG sites of specific genes.

A further object of the present invention is to provide a method for providing diagnostic information for prostate cancer, comprising the steps of measuring and comparing the methylation levels of CpG sites of specific genes from different samples.

Additionally, another object of the present invention is to provide the use of an agent for measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88 for the manufacture of a composition for diagnosing prostate cancer.

Furthermore, another object of the present invention is to provide a method for diagnosing prostate cancer comprising:
a) extracting a sample from a patient suspected of having prostate cancer;
b) measuring a methylation level of CpG sites of one or more genes selected from the group consisting of KLK4, HES5, TPM4, MOB3B, NNT, IL17RD, DTX4, NOL3, ADD3, EXT1, CPED1, and C2orf88 from the sample;
c) comparing the methylation level of the CpG sites of the gene(s) with those of a normal control sample; and
d) diagnosing prostate cancer if the methylation level of the CpG sites is increased compared to the normal control sample in step c).

### Solution for the Technical Problem

To achieve the above objectives, the present invention provides a composition for diagnosing prostate cancer comprising an agent for measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88.

Additionally, the present invention provides a composition for diagnosing prostate cancer consisting of an agent for measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88.

Furthermore, the present invention provides a composition for diagnosing prostate cancer consisting essentially of an agent for measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88.

To achieve another objective of the present invention, the present invention provides a diagnostic kit for prostate cancer comprising a primer pair for amplifying a fragment including the CpG sites of one or more genes selected from the group consisting of KLK4, HES5, TPM4, MOB3B, NNT, IL17RD, DTX4, NOL3, ADD3, EXT1, CPED1, and C2orf88.

To achieve another objective of the present invention, the present invention provides a nucleic acid chip for diagnosing prostate cancer, which has a probe fixed that can hybridize with a fragment containing the CpG site of one or more genes selected from the group consisting of KLK4, HES5, TPM4, MOB3B, NNT, IL17RD, DTX4, NOL3, ADD3, EXT1, CPED1, and C2orf88.

To achieve another objective of the present invention, the present invention provides a method for providing diagnostic information for prostate cancer, comprising the steps of
measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88 from a sample of a patient suspected of having prostate cancer; and
comparing the measured methylation level with the methylation level of the CpG sites of the same genes in a normal control sample.

To achieve another objective of the present invention, the present invention provides the use of an agent for measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88 for the manufacture of a composition for diagnosing prostate cancer.

Additionally, to achieve another objective of the present invention, the present invention provides a method for diagnosing prostate cancer comprising:
a) extracting a sample from a patient suspected of having prostate cancer;
b) measuring a methylation level of CpG sites of one or more genes selected from the group consisting of KLK4, HES5, TPM4, MOB3B, NNT, IL17RD, DTX4, NOL3, ADD3, EXT1, CPED1, and C2orf88 from the sample;
c) comparing the methylation level of the CpG sites of the gene(s) with those of a normal control sample; and
d) diagnosing prostate cancer if the methylation level of the CpG sites is increased compared to the normal control in step c).

Unless otherwise defined, all technical and scientific terms used herein have the same meaning as commonly understood by one of ordinary skill in the art. The following references provide one of skill with the general definition of many of the terms used in this specification of the present invention: Singleton et al., DICTIONARY OF MICROBIOLOGY AND MOLECULAR BIOLOGY (2nd ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY

### BRIEF DESCRIPTION OF THE DRAWINGS

FIG. 1 shows the methylation information of the KLK4 gene in a total of 24 types of cancer.

FIG. 2 shows the methylation information of the HES5 gene in a total of 24 types of cancer.

FIG. 3 shows the methylation information of the TPM4 gene in a total of 24 types of cancer.

FIG. 4 shows the methylation information of the MOB3B gene in a total of 24 types of cancer.

FIG. 5 shows the methylation information of the NNT gene in a total of 24 types of cancer.

FIG. 6 shows the methylation information of the IL17RD gene in a total of 24 types of cancer.

FIG. 7 shows the methylation information of the DTX4 gene in a total of 24 types of cancer.

FIG. 8 shows the methylation information of the NOL3 gene in a total of 24 types of cancer.

FIG. 9 shows the methylation information of the ADD3 gene in a total of 24 types of cancer.

FIG. 10 shows the methylation information of the EXT1 gene in a total of 24 types of cancer.

FIG. 11 shows the methylation information of the CPED1 gene in a total of 24 types of cancer.

FIG. 12 shows the methylation information of the C2orf88 gene in a total of 24 types of cancer.

FIG. 13 shows, as a comparative example, the methylation information of the TAMALIN gene in a total of 24 types of cancer.

FIG. 14 shows the diagnostic accuracy of prostate cancer using a total of 12 genes selected according to the present invention.

### DETAILED DESCRIPTION

Hereinafter, the present invention will be described in detail.

The present invention provides a composition for diagnosing prostate cancer, comprising an agent for measuring the methylation level of CpG sites of one or more genes selected from the group consisting of KLK4, HES5, TPM4, MOB3B, NNT, IL17RD, DTX4, NOL3, ADD3, EXT1, CPED1, and C2orf88.

In the present invention, “diagnosis of prostate cancer” preferably refers to the specific diagnosis of prostate cancer only. It does not diagnose or detect other cancers, such as bladder cancer, cervical cancer, bile duct cancer, colon cancer, esophageal cancer, glioblastoma, head and neck cancer, kidney chromophobe, kidney clear cell carcinoma, kidney papillary cell carcinoma, acute myeloid leukemia, liver hepatocellular carcinoma, lung adenocarcinoma, lung squamous cell carcinoma, mesothelioma, pancreatic adenocarcinoma, pheochromocytoma & paraganglioma, rectum adenocarcinoma, sarcoma, stomach adenocarcinoma, testicular cancer, thyroid cancer, thymoma, but specifically diagnoses or detects only prostate cancer.

Therefore, more preferably, “diagnosis of prostate cancer” in the present invention refers to detecting prostate cancer while not detecting bladder cancer, cervical cancer, bile duct cancer, colon cancer, esophageal cancer, glioblastoma, head and neck cancer, kidney chromophobe, kidney clear cell carcinoma, kidney papillary cell carcinoma, acute myeloid leukemia, liver hepatocellular carcinoma, lung adenocarcinoma, lung squamous cell carcinoma, mesothelioma, pancreatic adenocarcinoma, pheochromocytoma & paraganglioma, rectum adenocarcinoma, sarcoma, stomach adenocarcinoma, testicular cancer, thyroid cancer, thymoma.

In the present invention, the term “methylation” refers to the attachment of a methyl group to the bases that make up DNA. Preferably, in the present invention, the presence or absence of methylation refers to the occurrence of methylation at the cytosine of specific CpG sites of a specific gene. When methylation occurs, it interferes with the binding of transcription factors, thereby inhibiting the expression of specific genes. Conversely, when demethylation or hypomethylation occurs, the expression of specific genes is increased.

The genome DNA of mammalian cells contains a fifth base, 5-methylcytosine (5-mC), which is a cytosine with a methyl group attached to the fifth carbon of the cytosine ring, in addition to A, C, G, and T. Methylation of 5-methylcytosine occurs only at the C of the CG dinucleotide (5′-mCG-3′) referred to as CpG, and the methylation of CpG inhibits the expression of alu or transposons and repetitive sequences of the genome. Furthermore, the 5-mC of CpG is prone to spontaneous deamination, converting to thymine (T), making CpG sites hotspots for most epigenetic changes in mammalian cells.

In the present invention, the term “measurement of methylation level” refers to measuring the methylation level of CpG sites in one or more genes selected from the group consisting of KLK4, HES5, TPM4, MOB3B, NNT, IL17RD, DTX4, NOL3, ADD3, EXT1, CPED1, and C2orf88. This measurement can be performed using bisulfite-dependent or bisulfite-independent detection methods. The measurement of methylation level can be conducted through methylation-specific PCR methods, such as methylation-specific polymerase chain reaction (MSP), real-time methylation-specific polymerase chain reaction, PCR using methylation DNA-specific binding proteins, or quantitative PCR. Additionally, automated sequencing such as pyrosequencing and bisulfite sequencing can be used, though not limited to these methods. Furthermore, bisulfite-independent detection methods, such as the methods using TET proteins (ten-eleven translocation proteins), can be used to measure the methylation level. The TET proteins act on DNA and are involved in the chemical changes of bases; unlike bisulfite treatment, where all Cs except methylated Cs are converted to Ts, TET proteins convert only methylated Cs to Ts, enabling more efficient detection.

Preferably, the CpG sites of one or more genes selected from the group consisting of KLK4, HES5, TPM4, MOB3B, NNT, IL17RD, DTX4, NOL3, ADD3, EXT1, CPED1, and C2orf88 refer to the CpG sites present in the DNA of these genes. The DNA of these genes includes all the units necessary for their expression and operably linked, such as promoter regions, protein-coding regions (open reading frames, ORFs), and terminator regions. Therefore, the CpG sites of one or more genes selected from the group consisting of KLK4, HES5, TPM4, MOB3B, NNT, IL17RD, DTX4, NOL3, ADD3, EXT1, CPED1, and C2orf88 can be present in the promoter regions, protein-coding regions, or terminator regions of these genes.

Preferably, in the present invention, measuring the methylation level of the CpG sites of one or more genes selected from the group consisting of KLK4, HES5, TPM4, MOB3B, NNT, IL17RD, DTX4, NOL3, ADD3, EXT1, CPED1, and C2orf88 can refer to measuring the methylation level of cytosine at the CpG sites of the genes listed in Table 1 below.

TABLE 1

| Symbol | Genome Build | Chromosome | Region |
|---|---|---|---|
| KLK4 | GRCh37/hg19 | 19 | 51416002-51416249 |
| HES5 | GRCh37/hg19 | 1 | 2460760-2462010 |
| TPM4 | GRCh37/hg19 | 19 | 16186789-16188275 |
| MOB3B | GRCh37/hg19 | 9 | 27528976-27529885 |
| NNT | GRCh37/hg19 | 5 | 43602977-43603650 |
| IL17RD | GRCh37/hg19 | 3 | 57198243-57199378 |
| DTX4 | GRCh37/hg19 | 11 | 58940830-58941052 |
| NOL3 | GRCh37/hg19 | 16 | 67204208-67204637 |
| ADD3 | GRCh37/hg19 | 10 | 111767087-111768355 |
| EXT1 | GRCh37/hg19 | 8 | 119123974-119124432 |
| CPED1 | GRCh37/hg19 | 7 | 120628545-120628979 |
| C2orf88 | GRCh37/hg19 | 2 | 191044979-191045829 |

In the present invention, the CpG sites of one or more genes selected from the group consisting of KLK4, HES5, TPM4, MOB3B, NNT, IL17RD, DTX4, NOL3, ADD3, EXT1, CPED1, and C2orf88 are characterized by being located within +/−3000 bases (3 kb) from the transcription start site (TSS) of the gene.

In the present invention, the sequences of human genome chromosomal regions are represented according to ‘The February 2009 Human reference sequence (GRCh37/hg19)’. However, the designated sequences of these human genome chromosomal regions may be slightly modified as genome sequence research results are updated, and accordingly, numbering of the site of these human genome chromosomal regions in this invention may vary. Therefore, even if the human reference sequence is updated after the filing date of this invention and the number of the site of the human genome chromosomal regions changes, it is evident that the scope of this invention covers the updated human genome chromosomal regions. Such modifications are easily recognized by those skilled in the art to which this invention pertains.

In the present invention, the agent for measuring the methylation level of the CpG sites may comprise a compound that modifies cytosine bases or methylation-sensitive restriction enzymes, primers specific to the methylated allele sequences of one or more genes selected from the group consisting of KLK4, HES5, TPM4, MOB3B, NNT, IL17RD, DTX4, NOL3, ADD3, EXT1, CPED1, and C2orf88, and primers specific to the unmethylated allele sequences of above-mentioned genes.

The compound that modifies cytosine bases may be a compound that modifies unmethylated cytosine or methylated cytosine, and it may be, but not limited to, bisulfite or its salt, preferably sodium bisulfite, which modifies unmethylated cytosine, or TET proteins that modify methylated cytosine. Methods for detecting the methylation status of CpG sites by modifying cytosine bases are widely known in the art (WO01/26536; US2003/0148326A1).

Additionally, the methylation-sensitive restriction enzyme may be a restriction enzyme that specifically detects methylation of CpG sites, containing CG in the recognition site of the restriction enzyme. Examples include, but are not limited to, Smal, Sacll, Eagl, Hpall, Mspl, BssHll, BstUl, Notl. Depending on the methylation or unmethylation of the C in the recognition site of the restriction enzyme, the cutting by the restriction enzyme varies, and this can be detected through PCR or Southern Blot analysis. Other methylation-sensitive restriction enzymes are well known in the art.

The primers may include primers specific to the methylated allele sequences and primers specific to the unmethylated allele sequences of one or more genes selected from the group consisting of KLK4, HES5, TPM4, MOB3B, NNT, IL17RD, DTX4, NOL3, ADD3, EXT1, CPED1, and C2orf88.

In the present invention, the term “primer” refers to a short nucleic acid with a free 3′ hydroxyl end that can form base pairs with a complementary template and serves as a starting point for template strand replication. The primer, in the presence of appropriate buffer solution and temperature, as well as the necessary reagents for polymerization reactions (i.e., DNA polymerase or reverse transcriptase) and the four different nucleoside triphosphates, can initiate DNA synthesis. Additionally, the primer can incorporate additional features that do not alter its fundamental property of acting as an initiation point for DNA synthesis, as a sense or antisense nucleic acid with a sequence of 7 to 50 nucleotides.

The primer of the present invention can be preferably designed according to the sequence of a specific CpG site being analyzed for methylation status. More preferably, the primer can be selected from a group comprising primer pairs that can specifically amplify cytosine that is methylated and not modified by bisulfite, primer pairs that can specifically amplify cytosine that is unmethylated and modified by bisulfite, primer pairs that can specifically amplify cytosine that is methylated and modified by TET family proteins, and primer pairs that can specifically amplify cytosine that is unmethylated and not modified by TET family proteins.

Therefore, the present invention provides a kit for diagnosing prostate cancer, comprising primer pairs for amplifying fragments containing CpG sites of one or more genes selected from the group consisting of KLK4, HES5, TPM4, MOB3B, NNT, IL17RD, DTX4, NOL3, ADD3, EXT1, CPED1, and C2orf88.

The composition and kit may further comprise polymerase, agarose, buffer solutions necessary for electrophoresis, and other components in addition to the aforementioned agents.

The present invention also provides a nucleic acid chip for diagnosing prostate cancer, comprising probes fixed for hybridization with fragments containing CpG sites of one or more genes selected from the group consisting of KLK4, HES5, TPM4, MOB3B, NNT, IL17RD, DTX4, NOL3, ADD3, EXT1, CPED1, and C2orf88.

In the present invention, the term “nucleic acid” refers to oligonucleotides, nucleotides, polynucleotides, or their fragments; single-stranded or double-stranded DNA or RNA of genomic or synthetic origin; sense or antisense strands of genomic or synthetic origin; PNA (peptide nucleic acid); or natural or synthetic DNA or RNA molecules. It is apparent to those skilled in the art that if the nucleic acid is RNA, deoxynucleotides A, G, C, and T are replaced by ribonucleotides A, G, C, and U, respectively.

Since methylation begins at the periphery of the regulatory region of a gene and progresses inward, by detecting methylation at the periphery of the regulatory region, it is possible to diagnose early genes involved in cellular transformation.

Therefore, the use of these methylation gene markers enables the early diagnosis of cells that may form prostate cancer. If a gene confirmed to be methylated in cancer cells is also methylated in cells that appear clinically or morphologically normal, it indicates that these seemingly normal cells are undergoing carcinogenesis. Thus, by confirming the methylation of prostate cancer-specific genes in seemingly normal cells, prostate cancer can be diagnosed at an early stage.

Furthermore, the present invention provides a method for providing information for diagnosing prostate cancer, comprising: measuring methylation levels of CpG sites of one or more genes selected from the group consisting of KLK4, HES5, TPM4, MOB3B, NNT, IL17RD, DTX4, NOL3, ADD3, EXT1, CPED1, and C2orf88 from a sample of a patient suspected of having prostate cancer; and comparing the measured methylation levels with the methylation levels of the same CpG sites in a normal control sample.

The method for measuring the methylation level can be selected from the group consisting of, but not limited to, PCR, methylation-specific PCR, real-time methylation-specific PCR, PCR using methylation-specific DNA binding proteins, methylation status measurement using methylation-sensitive restriction enzymes, quantitative PCR, DNA chip, pyrosequencing, and bisulfite sequencing.

Specifically, the method of methylation-specific PCR (methylation specific PCR) involves treating sample DNA with bisulfite and then using primers designed based on the methylation status of CpG dinucleotides for PCR. If the primer binding site is methylated, PCR proceeds with the primer specific to methylation, and if it is unmethylated, PCR proceeds with the primer specific to unmethylation. This method involves treating the sample DNA with bisulfite, using two types of primers simultaneously for PCR, and then comparing the results.

Real-time methylation-specific PCR converts the methylation-specific PCR method into a real-time method. After treating genomic DNA with bisulfite, PCR primers corresponding to the methylated regions are designed and real-time PCR is performed using these primers. Detection can be done using either TanMan probes complementary for the amplified sequences or Sybergreen. Therefore, real-time methylation-specific PCR can selectively quantify methylated DNA. A standard curve is created using in vitro methylated DNA samples, and a gene sequence without a 5′-CpG-3′ sequence is amplified as a negative control to quantify the degree of methylation.

In the method of measuring methylation using methylation-sensitive restriction enzymes, the enzymes act on CpG dinucleotides and do not function if the site is methylated. Therefore, after treating the sample DNA with methylation-sensitive restriction enzymes, PCR amplification is performed to include the enzyme target site. If the site is methylated, the enzyme does not act and PCR proceeds, but if the site is unmethylated, it is cut by the enzyme and PCR does not proceed, allowing the measurement of the methylation status of specific DNA regions.

The PCR or DNA chip method using methylation-specific binding proteins involves mixing proteins that specifically bind only to methylated DNA with whole DNA, thus selectively isolating only methylated DNA. After mixing genomic DNA with methylation-specific binding proteins, only the methylated DNA is selectively isolated. The isolated DNA is amplified using PCR primers corresponding to intron regions, and the methylation status is measured by agarose gel electrophoresis. Additionally, methylation can be measured by quantitative PCR, and the methylated DNA isolated by methylation-specific binding proteins can be labeled with fluorescent dyes and hybridized to a DNA chip with complementary probes. The methylation-specific binding proteins are not limited to MBD2bt.

Bisulfite pyrosequencing of bisulfite-treated DNA is based on the following principle. When methylation occurs at a CpG dinucleotide site, 5-methylcytosine (5-mC) is formed, and this modified base is converted to uracil when treated with bisulfite. If CpG dinucleotides are methylated, cytosine is preserved when treated with bisulfite to DNA extracted from sample, while unmethylated cytosine is converted to uracil. Sequencing of bisulfite-treated DNA can preferably be performed using the pyrosequencing method, details of which are well known in the art.

Meanwhile, bisulfite-independent detection using TET proteins involves using TET proteins to convert methylated cytosines to thymines, thus detecting the base at the methylated site.

When 5-methylcytosine (5-mC) is formed due to methylation at CpG dinucleotide sites, treatment with TET (ten-eleven translocation) proteins converts methylated CpG dinucleotides to uracil, while unmethylated cytosine remains unchanged. Sequence analysis of TET-treated DNA is not limited to pyrosequencing and can be performed using methods such as methylation-sensitive PCR (MSP), microarray, or next generation sequencing (NGS).

Preferably, the method for providing information for diagnosing prostate cancer in the present invention comprises the steps of: a) obtaining a sample from an subject, b) extracting genomic DNA from the sample, c) treating the extracted genomic DNA with a compound that modifies unmethylated cytosine bases, d) amplifying the treated DNA by PCR using primers capable of amplifying one or more CpG sites selected from the group consisting of KLK4, HES5, TPM4, MOB3B, NNT, IL17RD, DTX4, NOL3, ADD3, EXT1, CPED1, and C2orf88 to obtain PCR products, and e) measuring the degree of methylation of the PCR products.

The genomic DNA extraction in step b) can be performed using methods commonly used in the art, such as phenol/chloroform extraction, SDS extraction, CTAB separation, or commercially available DNA extraction kits.

In the present invention, the term “sample” refers to a wide range of biological fluids obtained from subject, body fluids, cell lines, and tissue cultures depending on the type of analysis being performed. Methods for obtaining body fluids and tissue biopsies from mammals are widely known, and in the present invention, the sample preferably comprises human-derived materials selected from the group consisting of tissues, cells, blood, plasma, serum, stool, and urine. Abnormal methylation changes in cancer tissues show significant similarity to methylation changes in genomic DNA obtained from biological samples such as cells, blood, serum, plasma, saliva, sputum, cerebrospinal fluid, or urine. Thus, the use of the markers of the present invention has the advantage of allowing easy diagnosis of prostate cancer through blood, body fluids and so on.

Additionally, the present invention provides the use of an agent for measuring the methylation level of the CpG site of one or more genes selected from the group consisting of KLK4, HES5, TPM4, MOB3B, NNT, IL17RD, DTX4, NOL3, ADD3, EXT1, CPED1, and C2orf88 for the preparation of a composition for diagnosing prostate cancer.

The present invention also provides a method for diagnosing prostate cancer comprising:
a) extracting a sample from a patient suspected of having prostate cancer;
b) measuring the methylation level of the CpG site of one or more genes selected from the group consisting of KLK4, HES5, TPM4, MOB3B, NNT, IL17RD, DTX4, NOL3, ADD3, EXT1, CPED1, and C2orf88 from the sample;
c) comparing the methylation level of the CpG site of the gene with that of a normal control sample; and
d) diagnosing prostate cancer if the methylation level of the CpG site is increased compared to the normal control sample in step c).

In one embodiment, the present invention provides a method for diagnosing and treating prostate cancer in an subject comprising the steps of:
i) extracting a sample from a patient suspected of having prostate cancer;
ii) measuring the methylation level of the CpG site of one or more genes selected from the group consisting of KLK4, HES5, TPM4, MOB3B, NNT, IL17RD, DTX4, NOL3, ADD3, EXT1, CPED1, and C2orf88 from the sample;
iii) comparing the methylation level of the CpG site of the gene with that of a normal control sample;
iv) diagnosing prostate cancer if the methylation level of the CpG site is increased compared to the normal control sample in step iii); and
v) treating the diagnosed subject with a therapeutic drug or surgery to treat the prostate cancer.

The methods comprising steps i) to v) are understood in accordance with the methods comprising steps a) to d) described above.

Step v) involves treating the disease diagnosed in step iv) by administering therapeutic drugs such as enzalutamide, relugolix, apalutamide, and darolutamide, or by performing surgical procedures.

The term “effective amount” in the present invention refers to an amount that, when administered to a subject, shows an effect of improving, treating, detecting, diagnosing, or inhibiting or reducing prostate cancer or its symptoms. The term “subject” can refer to animals, preferably mammals, particularly humans, and can include cells, tissues, or organs derived from animals. The subject may be a patient in need of the effect.

The term “treatment” in the present invention comprehensively refers to improving symptoms caused by prostate cancer or the disease, and can include curing, substantially preventing, or improving the condition, and includes alleviating, curing, or preventing one or more symptoms or most symptoms caused by the disease, but is not limited thereto.

In this specification, the term “comprising” is used interchangeably with “including” or “characterized by” and does not exclude additional components or method steps not specifically mentioned in the composition or method according to the present invention. The term “consisting of” excludes any additional elements, steps, or components not specified. The term “consisting essentially of” means that the composition or method can include materials or steps that do not materially affect the basic characteristics of the composition or method.

### Advantageous Effects of Invention

As discussed above, hypermethylation of CpG sites of one or more genes selected from the group consisting of KLK4, HES5, TPM4, MOB3B, NNT, IL17RD, DTX4, NOL3, ADD3, EXT1, CPED1, and C2orf88 is specific for prostate cancer, and thus, the compositions, kits, chips, or methods according to the present invention can be used to accurately and rapidly diagnose prostate cancer, and furthermore, to diagnose prostate cancer at an early stage.

### Mode of Operation

The following preferred embodiments are presented to aid in the understanding of the present invention. However, these embodiments are provided merely to facilitate comprehension of the invention and are not intended to limit the scope of the invention.

### Example 1: Screening of Prostate Cancer-Specific Methylated Genes

To identify methylated genes specific to prostate cancer, a large-scale methylation comparison study was conducted using data from two large-scale methylation microarray chips and comparing cancer tissues obtained from prostate cancer surgeries with normal tissues (refer to Table 2). In this study, tumor tissue refers to prostate cancer tissues, while non-tumor tissue includes normal prostate tissues and other non-cancerous tissues.

TABLE 2
<Datasets used for screening prostate
cancer-specific methylated regions>

| | dataset #1 | dataset #2 | total |
|---|---|---|---|
| Tumor | n = 112 | n = 395 | n = 507 |
| non-tumor | n = 149 | n = 45 | n = 194 |

To screening prostate cancer-specific methylated genes, DNA was extracted from each tissue, and the level of methylation in gene regions was determined by using the Infinium Human Methylation 450 Beadchip microarray.

The DNA extracted from each tissue was treated by bisulfite treatment, which modifies cytosine bases depending on the methylation status. Probes used in this microarray experiment were designed to specifically detect methylated and unmethylated cytosine bases in gene regions.

The microarray experiment measured the degree of methylation in genes using approximately 450,000 (450 k) probes representing methylated regions, with results presented as beta values. The beta value ranges from 0 to 1, with higher values indicating higher methylation levels of the gene region.

To identify differentially methylated regions (DMRs) between tumor and non-tumor groups, the empirical Bayes t-test method known as Limma (Linear Models for Microarray Data) was used to identify gene regions with statistically significant methylation differences between groups.

The Limma method is known to be less affected by outliers compared to other methylation statistical analysis methods, making it suitable for finding cancer-specific markers. In this experiment, a lower adjusted p-value derived from the Limma method indicated a significant methylation difference between the two groups.

Particularly, to screen for tumor-specific methylated regions, gene regions with significant beta value differences between tumor and non-tumor groups and with higher methylation levels in tumor tissues compared to non-tumor tissues, were selected as cancer-specific biomarker candidates.

As a result, Limma analysis from both datasets identified gene regions with significantly lower p-values and higher average methylation levels in tumor groups compared to non-tumor groups, designating these as tumor-specific hypermethylated regions. From approximately 450,000 gene regions, 8,089 gene regions common to both datasets exhibiting tumor-specific hypermethylation were selected as biomarker candidates.

### Example 2: Identification of Prostate Cancer-Specific Hypermethylated Genes

From the 8,089 gene regions identified as biomarkers in Example 1, methylation levels of each region were confirmed and compared in tumors other than prostate cancer to find gene regions specific to prostate cancer. Analysis of DNA methylation 450 k array experimental results from the TCGA (The Cancer Genome Atlas), a public cancer gene database, confirmed the methylation information of gene regions corresponding to major cancer types. From these, 202 gene regions showing significantly higher beta values in prostate cancer compared to the other 23 major cancer types, excluding prostate adenocarcinoma, were identified as prostate cancer-specific methylated regions.

Among these gene regions, 1) non-pseudogenes, 2) gene regions with significant methylation differences between tumor and non-tumor tissues (large delta p value), 3) regions within CpG islands, 4) regions within +/−3000 bases (3 kb) from the transcription start site (TSS), and 5) regions located on autosomes were selected as prostate cancer-specific hypermethylated genes. As a result, a total of 12 genes were selected as shown in Table 3.

TABLE 3
<Prostate Cancer-Specific Gene Regions>

| | | CpG island | Delta β | Delta β |
|---|---|---|---|---|
| Symbol | Name | (GRCh37/hg19) | (Dataset #1) | (Dataset #2) |
| KLK4 | kallikrein related | chr19: 51416002- | 0.61 | 0.53 |
| | peptidase 4 | 51416249 | | |
| HES5 | hes family bHLH | chr1: 2460760- | 0.60 | 0.42 |
| | transcription factor 5 | 2462010 | | |
| TPM4 | tropomyosin 4 | chr19: 16186789- | 0.58 | 0.49 |
| | | 16188275 | | |
| MOB3B | MOB kinase | chr9: 27528976- | 0.45 | 0.42 |
| | activator 3B | 27529885 | | |
| NNT | nicotinamide nucleotide | chr5: 43602977- | 0.68 | 0.46 |
| | transhydrogenase | 43603650 | | |
| IL17RD | interleukin 17 | chr3: 57198243- | 0.57 | 0.45 |
| | receptor D | 57199378 | | |
| DTX4 | deltex E3 ubiquitin | chr11: 58940830- | 0.66 | 0.51 |
| | ligase 4 | 58941052 | | |
| NOL3 | nucleolar protein 3 | chr16: 67204208- | 0.54 | 0.45 |
| | | 67204637 | | |
| ADD3 | adducin 3 | chr10: 111767087- | 0.49 | 0.39 |
| | | 111768355 | | |
| EXT1 | exostosin glycosyltransferase 1 | chr8: 119123974- | 0.50 | 0.38 |
| | | 119124432 | | |
| CPED1 | cadherin like and PC- | chr7: 120628545- | 0.39 | 0.31 |
| | esterase domain containing 1 | 120628979 | | |
| C2orf88 | chromosome 2 open reading | chr2: 191044979- | 0.58 | 0.45 |
| | frame 88 | 191045829 | | |

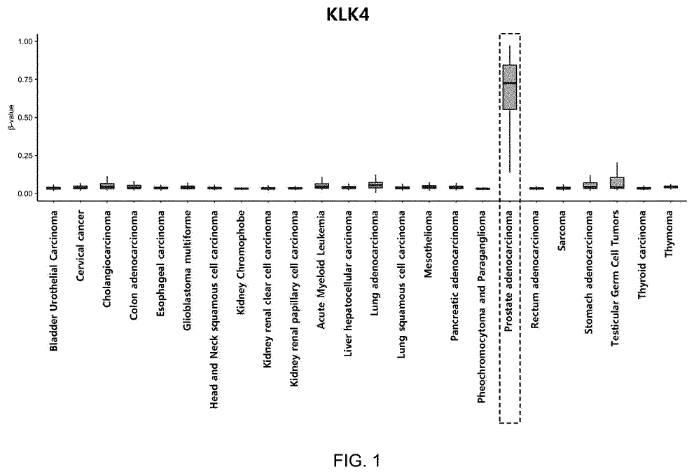

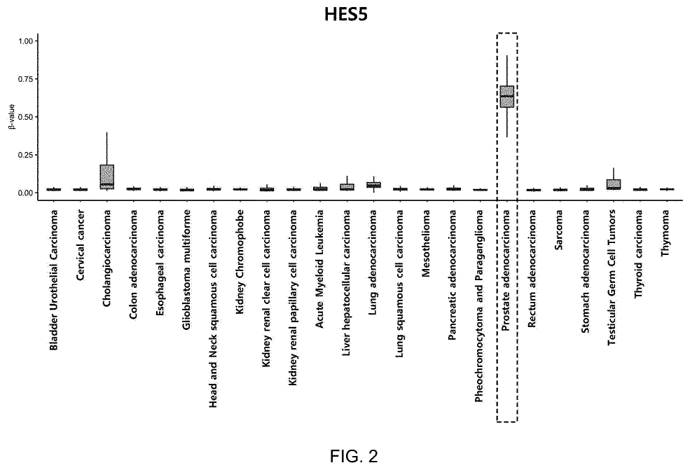

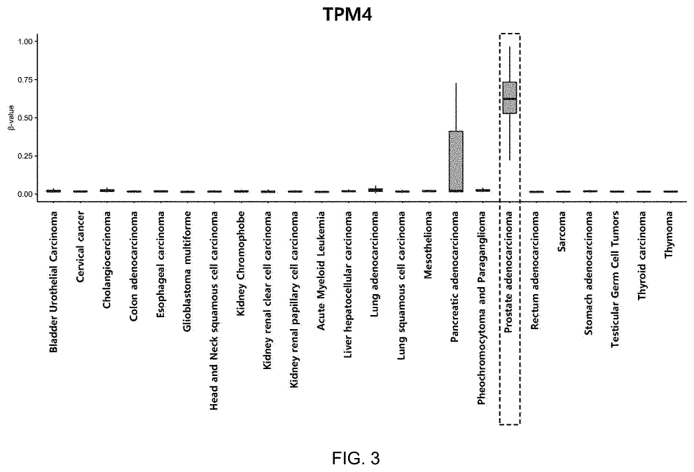

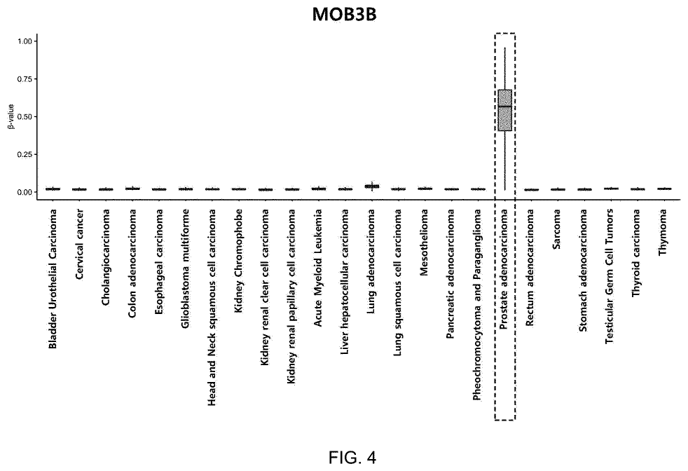

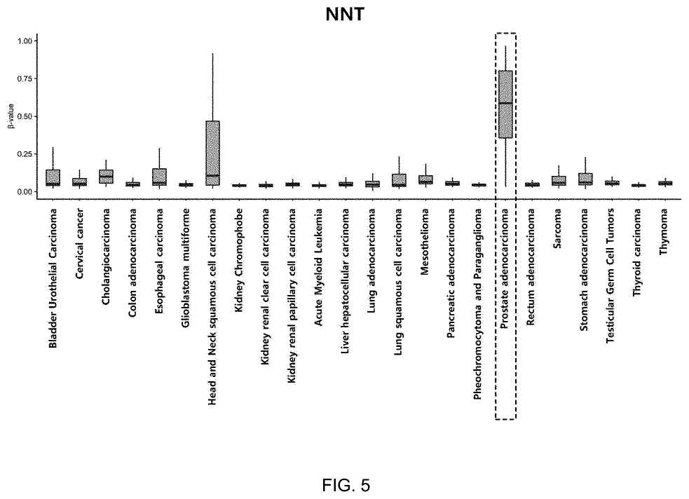

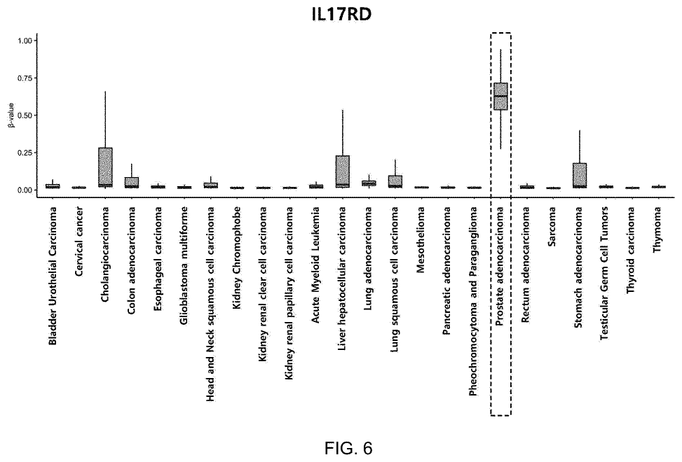

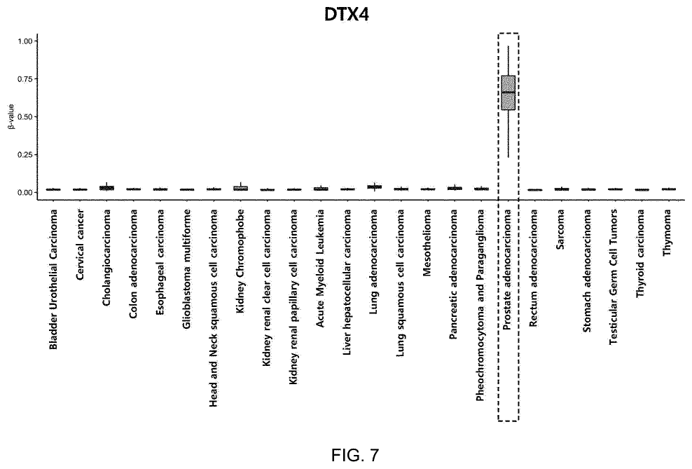

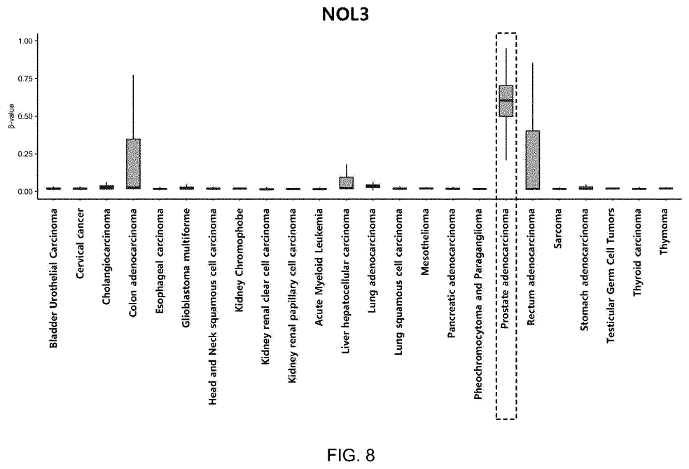

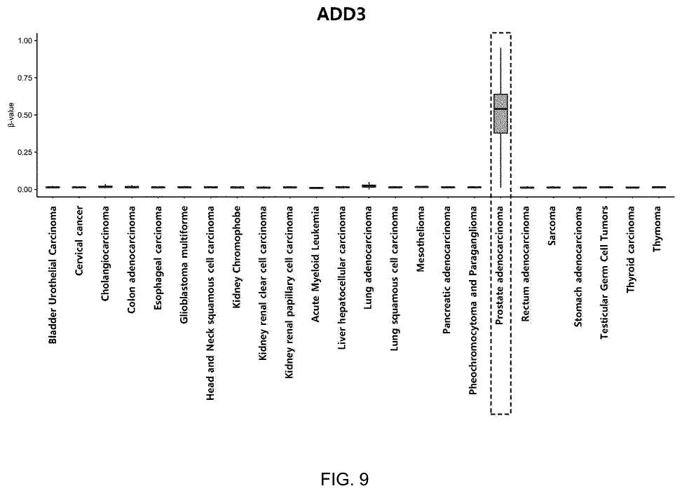

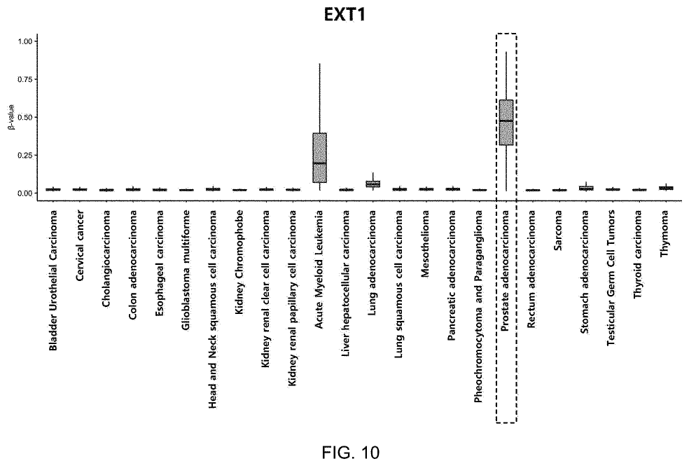

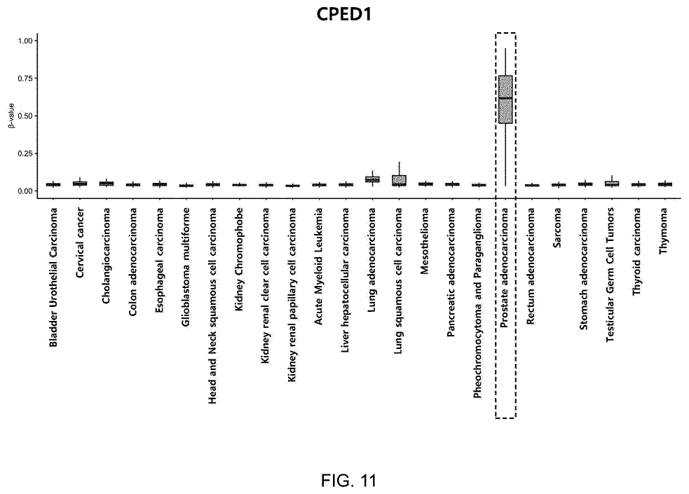

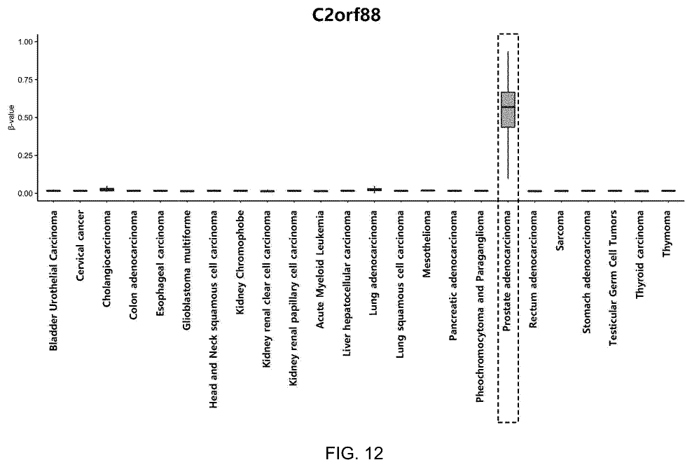

The degree of methylation of these genes in tumor tissue (prostate cancer tissue) and non-tumor tissue (other non-cancerous tissues including normal prostate tissue) through microarray experiments is shown in FIGS. 1 to 12. The degree of methylation is represented by beta values obtained from the experiment, with beta values ranging from 0 to 1, and higher values indicating higher methylation levels of the gene region.

Meanwhile, although significant methylation differences were observed in gene regions when comparing prostate cancer tumor tissues with non-tumor tissues, this did not confirm prostate cancer-specific methylation. Methylation can also be observed in other cancers.

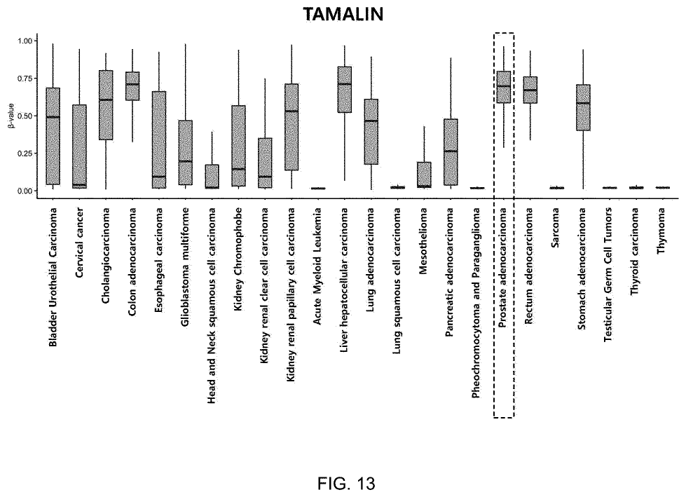

For example, TAMALIN (trafficking regulator and scaffold protein tamalin) gene showed one of the largest methylation differences between tumor and non-tumor tissues in prostate tissue among the 8,089 gene regions identified in Example 1. However, as shown in FIG. 13, high methylation was also observed in various cancers including colon cancer, rectum cancer, liver cancer, and stomach cancer.

The 23 cancers other than prostate cancer are as follows:

Bladder cancer, Cervical Cancer, Bile Duct cancer, Colon cancer, Esophageal Cancer, Glioblastoma, Head and neck cancer, Kidney chromophobe, Kidney Clear cell carcinoma, Kidney Papillary cell carcinoma, Acute myeloid leukemia, Liver hepatocellular carcinoma, Lung adenocarcinoma, Lung squamous cell carcinoma, Mesothelioma, Pancreatic adenocarcinoma, Pheochromocytoma & paraganglioma, Rectum adenocarcinoma, Sarcoma, Stomach adenocarcinoma, Testicular cancer, Thyroid cancer, Thymoma.

### Example 3: Evaluation of Diagnostic Accuracy of Prostate Cancer Diagnostic Marker Candidates

To verify the performance of the selected genes as diagnostic markers in prostate cancer, the diagnostic accuracy of prostate cancer based on the degree of methylation was evaluated.

Sensitivity and specificity are used to assess the accuracy of a diagnostic test. The sensitivity and specificity values for potential cut-off points in consecutive diagnostic test measurements can be calculated to present the changes in sensitivity and specificity based on these cut-off points, forming a Receiver Operating Characteristic (ROC) curve. The accuracy of the diagnosis can be measured by the area under the ROC curve (AUC). The AUC value ranges from 0.5 to 1, with higher values indicating greater diagnostic accuracy. An AUC value of 1 indicates a perfectly accurate test, while a value of 0.5 indicates results equivalent to random chance.

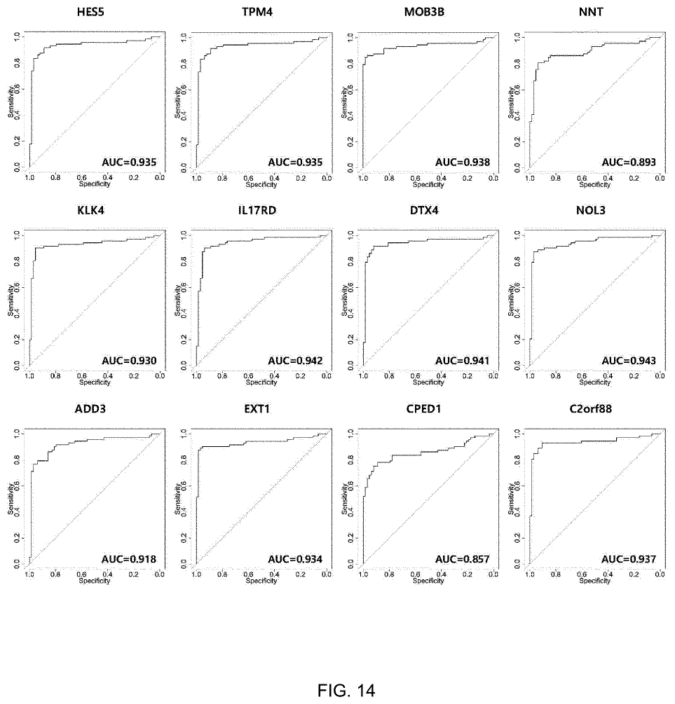

Analysis of the accuracy of cancer diagnosis based on the degree of methylation between non-tumor and tumor tissues using the selected genes, with both the patient group analyzed in the above example and an independent patient group, confirmed that all selected genes had AUC values of at least 0.857, demonstrating high diagnostic accuracy as shown in FIG. 14. Thus, the selected genes were verified to be useful for prostate cancer diagnosis.

### Example 4: Measurement of Methylation Based on qMSP and Digital PCR in Prostate Cancer Cell Lines and Tissue Samples

To verify the applicability of the selected genes as diagnostic tests in prostate cancer, the presence of specific methylation in prostate cancer was confirmed using a PCR (quantitative methylation-specific PCR, qMSP) technique in a prostate cancer cell line (LNCaP clone FGC). For this purpose, PCR primers and probes were designed for the genetic region specified in [Table 3] in the prostate cancer cell line (LNCaP clone FGC), and detection performance was evaluated in the prostate cancer cell line and prostate cancer tissue using the qMSP detection method.

Genomic DNA was isolated from cancer samples and treated with bisulfite, then the methylation level of selected biomarkers was observed using a generalized qMSP experimental method. Additionally, the ACTB gene, unrelated to methylation, was used to measure the amplified value of general genomic DNA for standardization. The methylation level of the biomarker was measured using the general real-time PCR method.

The methylation level obtained by amplifying the bisulfite-converted DNA by PCR is expressed as ΔCt, which is a value corrected by the Ct (Cycle of Threshold) value of ACTB used as an internal control. ΔCt is defined as:

ΔCt=Ct value of the ACTB gene−Ct value of the target gene.

The qMSP method detected methylation sites in all 12 genes, showing significant amplification of methylation sites, confirming the presence of methylation in these genetic regions in prostate cancer.

Similarly, using digital PCR, which has higher detection sensitivity than real-time PCR, the prostate cancer-specific methylation level of the selected genes was observed. The bisulfite-modified genetic regions were amplified by PCR, using the ACTB gene as the internal control.

The methylation level of bisulfite-converted DNA was calculated as copy values using droplet digital PCR (ddPCR).

The results confirmed high methylation levels in all 12 genes in prostate cancer cell lines, similar to qMSP results.

Additionally, in prostate cancer tissues and in tissues from liver cancer, gastric cancer, colorectal cancer, and pancreatic cancer, methylation levels of the biomarkers were detected. Prostate cancer tissues showed high methylation levels above 100 copies, while other cancers showed low methylation levels around 10 copies, indicating the specificity of the selected genetic regions for prostate cancer.

These results demonstrate that the selected genes can be diagnosed using various molecular biological techniques.

Consequently, all 12 selected biomarkers were detected in cell lines and prostate cancer tissues, confirming that the selected genes can be detected using molecular diagnostic techniques for prostate cancer diagnosis.

### Example 5: Validation Using Blood and Urine Samples for Prostate Cancer Diagnostic Markers

As cell-free DNA (cfDNA) fragments from various tissues and organs circulate in the blood, determining the origin of cfDNA can identify the presence of cancer. This study verified whether the selected prostate cancer biomarkers can be utilized as liquid biopsy-based cancer diagnostic tests. The KLK4 gene was further validated.

Since most cfDNA in blood is known to originate from blood cells, the methylation level of KLK4 in blood cells was determined. Peripheral blood was collected from 10 healthy individuals, and genomic DNA was isolated from blood cells. A ddPCR-based molecular diagnostic test was performed to determine the methylation level of the KLK4 genetic region. The results showed low KLK4 methylation levels below 5 copies in all 10 individuals.

Additionally, plasma samples from 10 healthy individuals purchased from a biobank were tested using a ddPCR-based molecular diagnostic test to confirm the presence of KLK4 methylation. All 10 individuals showed ACTB gene values above 1,000 copies, indicating various DNA fragments in plasma samples, but KLK4 biomarker values were below 5 copies, confirming that high KLK4 methylation does not occur in healthy individuals.

These results show that KLK4 is a prostate cancer-specific methylation biomarker, with very low levels in the blood of healthy individuals and other cancers, indicating a low risk of false positives when using this biomarker for prostate cancer diagnosis.

To test the possibility of detecting prostate cancer in blood samples, DNA from the prostate cancer cell line LNCaP was mixed at various concentrations in normal plasma to observe the proportional detection of KLK4 according to concentration gradients. The results showed a linear increase in KLK4 copy values with increasing amounts of prostate cancer cell line-derived DNA. This indicates that the presence of prostate cancer-derived DNA in the blood can be detected using a molecular diagnostic test for KLK4.

Given the possibility of detecting prostate cancer-derived DNA fragments not only in blood but also in urine, the specificity of prostate cancer biomarkers was validated in urine samples. DNA was extracted from 10 ml urine samples collected from 5 healthy individuals without prostate cancer and pre-treated with bisulfite. A digital PCR-based biomarker detection experiment for KLK4 was performed, showing very low methylation levels below copies, similar to the blood test results, confirming that high KLK4 methylation does not occur in healthy individuals.

These results indicate that the selected prostate cancer-specific biomarkers also show specificity in other body fluids besides blood, suggesting their potential use in liquid biopsy tests.

### INDUSTRIAL APPLICABILITY

As discussed above, hypermethylation of the CpG site of one or more genes selected from the group consisting of KLK4, HES5, TPM4, MOB3B, NNT, IL17RD, DTX4, NOL3, ADD3, EXT1, CPED1, and C2orf88 occurs specifically in prostate cancer. Using the composition, kit, chip, or method according to the present invention, prostate cancer can be diagnosed not only accurately and quickly, but also at an early stage.

We claim:

1. A composition for diagnosing prostate cancer comprising an agent for measuring the methylation level of CpG sites in one or more genes selected from the group consisting of KLK4, HES5, TPM4, MOB3B, NNT, IL17RD, DTX4, NOL3, ADD3, EXT1, CPED1, and C2orf88.

2. The composition of claim 1, wherein the CpG sites are located within +/−3000 bases (3 kb) of the transcription start site of the gene.

3. The composition of claim 1, wherein the agent for measuring the methylation level of the CpG sites comprises:
a compound modifying unmethylated cytosine or methylated cytosine bases;
a primer specific to the methylated sequence of CpG sites in one or more genes selected from the group consisting of KLK4, HES5, TPM4, MOB3B, NNT, IL17RD, DTX4, NOL3, ADD3, EXT1, CPED1, and C2orf88; and
a primer specific to the unmethylated sequence.

4. The composition of claim 3, wherein the compound modifying unmethylated cytosine bases is bisulfite or a salt thereof, and the compound modifying methylated cytosine bases is a TET protein.

5. A kit for diagnosing prostate cancer comprising a primer pair for amplifying a fragment containing CpG sites in one or more genes selected from the group consisting of KLK4, HES5, TPM4, MOB3B, NNT, IL17RD, DTX4, NOL3, ADD3, EXT1, CPED1, and C2orf88.

6. A nucleic acid chip for diagnosing prostate cancer, comprising a probe fixed to hybridize with a fragment containing CpG sites in one or more genes selected from the group consisting of KLK4, HES5, TPM4, MOB3B, NNT, IL17RD, DTX4, NOL3, ADD3, EXT1, CPED1, and C2orf88.

7. A method for providing diagnostic information for prostate cancer, comprising:
measuring a methylation level of CpG site in one or more genes selected from the group consisting of KLK4, HES5, TPM4, MOB3B, NNT, IL17RD, DTX4, NOL3, ADD3, EXT1, CPED1, and C2orf88 from a sample of a patient suspected of having prostate cancer; and
comparing the measured methylation level with the methylation level of the same CpG sites in a normal control sample.

8. The method of claim 7, wherein the method for measuring the methylation level is selected from the group consisting of bisulfite-free detection methods, methylation-specific polymerase chain reaction, real-time methylation-specific polymerase chain reaction, PCR using methylated DNA-specific binding proteins, quantitative PCR, pyrosequencing, and bisulfite sequencing.

9. The method of claim 7, wherein the sample is selected from the group consisting of tissue, cells, blood, plasma, serum, stool, and urine.

10. Use of an agent for measuring the methylation level of CpG sites in one or more genes selected from the group consisting of KLK4, HES5, TPM4, MOB3B, NNT, IL17RD, DTX4, NOL3, ADD3, EXT1, CPED1, and C2orf88 for manufacturing a composition for diagnosing prostate cancer.

11. The use of claim 10, wherein the CpG sites are located within +/−3000 bases (3 kb) of the transcription start site of the gene.

12. The use of claim 10, wherein the agent for measuring the methylation level of CpG sites comprises:
a compound modifying unmethylated cytosine or methylated cytosine bases;
a primer specific to the methylated sequence of CpG sites in one or more genes selected from the group consisting of KLK4, HES5, TPM4, MOB3B, NNT, IL17RD, DTX4, NOL3, ADD3, EXT1, CPED1, and C2orf88; and
a primer specific to the unmethylated sequence.

13. A method for diagnosing prostate cancer comprising:
a) extracting a sample from a patient suspected of having prostate cancer;
b) measuring the methylation level of CpG sites in one or more genes selected from the group consisting of KLK4, HES5, TPM4, MOB3B, NNT, IL17RD, DTX4, NOL3, ADD3, EXT1, CPED1, and C2orf88 from the sample;
c) comparing the methylation level of CpG sites in the gene with a normal control sample; and
d) diagnosing prostate cancer if the methylation level of CpG sites is increased compared to the normal control in step c).

14. The method of claim 13, wherein the CpG sites are located within +/−3000 bases (3 kb) of the transcription start site of the gene.

15. The method of claim 13, wherein the method for measuring the methylation level is selected from the group consisting of bisulfite-free detection methods, methylation-specific polymerase chain reaction, real-time methylation-specific polymerase chain reaction, PCR using methylated DNA-specific binding proteins, quantitative PCR, pyrosequencing, and bisulfite sequencing.
